(19) United States
(12) Patent Application Publication
(10) Pub. No.: US 2018/0042891 A1
(43) Pub. Date: Feb. 15, 2018

(54) Cancer Cell Apoptosis

(71) Applicant: e-Therapeutics plc, Oxfordshire (GB)

(72) Inventors: Malcolm Philip Young, Northumberland (GB); Philip McKeown, Newcastle Upon Tyne (GB)

(73) Assignee: e-Therapeutics plc, Oxfordshire (GB)

(21) Appl. No.: 15/723,450

(22) Filed: Oct. 3, 2017

Related U.S. Application Data
(62) Division of application No. 13/390,832, filed on Apr. 6, 2012, now abandoned.

(30) Foreign Application Priority Data
Sep. 10, 2009 (GB) 0915877.5

(51) Int. Cl. A61K 31/352

(52) U.S. Cl. CPC A61K 31/352

(57) ABSTRACT

There is described a therapeutic agent capable of directly or indirectly having an effect on the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, simultaneously, sequentially or separately.

There is especially described dexanabinol, or a derivative thereof, as the therapeutic agent.

### CROSS REFERENCE TO RELATED APPLICATIONS

This application is a divisional application of U.S. patent application Ser. No. 13/390,832, filed on Apr. 6, 2012, which is a national phase application under 35 U.S.C. §371 of International Application No. PCT/GB2010/001710, filed on Sep. 10, 2010, which claims priority to and the benefit of GB Patent Application No. 0915877.5, filed on Sep. 10, 2009, the entire disclosures of each of which are incorporated by reference herein.

### FIELD OF THE INVENTION

The present invention provides medicaments and methods for the treatment of cancer and especially a therapy which provides apoptosis of cancer cells. More particularly the invention provides dexanabinol, or a derivative thereof, for the treatment of cancers other than melanoma, by apoptosis.

### BACKGROUND

Dexanabinol is 1, 1 dimethyl heptyl-(3S,4S)-7-hydroxy-Δ⁶-tetrahydrocannabinol which is disclosed in U.S. Pat. No. 4,876,276. Dexanabinol is a non psychotropic cannabinoid which has been previously demonstrated to rapidly kill melanoma cells in vitro.

International Patent application WO 2009/007700 describes the use of dexanabinol in the treatment of melanoma cancer cells. The apoptotic effect of dexanabinol is described, but the mechanism of action is not disclosed and was not fully understood at that time. Thus the applicability of the drug for use in other cancer cells other than melanoma was not previously foreseeable. In this previous application it has been disclosed that dexanabinol acts via inhibiting Nuclear Factor Kappa-B (NFκB) in a melanoma cell and thus provides a treatment for melanoma. Furthermore, it has been shown that in melanoma dexanabinol both induces apoptosis and inhibits cell proliferation.

We have since found that the mechanism of action of dexanabinol is more complex than just via binding to NFκB. The inventive step over that contemplated in WO '700 is the innovation of establishing the additional forms of cancer that dexanabinol induces apoptosis in as a result of the new knowledge of the mechanism of action. There is a complex profile of bindings as well as other indirect effects. This has led us to understand that dexanabinol is unexpectedly functional in more cancers than just melanoma and that it furthermore has a desirable selective apoptotic effect. We have now surprisingly found that dexanabinol is not only efficacious in melanoma but also in several other cancers.

The hitherto undisclosed binding profile and indirect effects of dexanabinol indicate that it would be effective at inducing apoptosis in other forms of cancer than just melanoma. From our investigations we have now established those cancers which are susceptible to apoptosis induction by dexanabinol as a result of the new knowledge regarding its mode of action. Based on this, relevant cell lines have been tested and the hypothesis has been confirmed.

International Patent application No. WO 03/077832 describes the use of dexanabinol in reducing cancer cell proliferation. Moreover, this decrease in proliferation is described with respect to regulation of inflammation related genes.

WO '832 only provides enabling evidence for pancreatic tumours and colorectal tumours. The experimental results show that “Aspc-1 proliferation was not affected by the presence of dexanabinol up to 151M whereas Panc-1 cells proliferation was inhibited by 26% at this same concentration”. It is also stated that dexanabinol “which acts through modulation of pro/anti-inflammatory mediators, may be therapeutically effective against certain types of tumours”.

Thus, the use of dexanabinol as a cancer treatment is disclosed, but it will be understood by the person skilled in the art that a reduction in cell proliferation may reduce the impact of a cancer by preventing it from spreading or growing but will not be fatal to the cancer itself and therefore may rely upon, for example, surgical techniques or other chemotherapy to cause the cancer to undergo cell apoptosis.

However, although dexanabinol has an effect on inflammation and thus cell proliferation, there is nothing to suggest that it would also have any apoptotic effect.

WO '832 recognises that the mechanism of action of dexanabinol is not well understood. Indeed it states, at page 1, lines 24 to 25, “Nevertheless, the mechanism underlying some therapeutic effects of cannabinoid derivatives remain unclear.” Furthermore, WO '832 describes that dexanabinol and other cannabinoids would be an attractive candidate for the treatment of neurological damage resulting from spinal chord injury, cerebral ischaemia and neurodegenerative disorders, such as Alzheimer's and Parkinson's diseases. As such, it would be readily appreciated that any apoptotic affect in these cannabinoids would be undesirable.

This art implicates dexanabinol in the treatment of these cancers, however not via an induction of apoptosis. This induction of selective apoptosis is key and is not anticipated by this art.

### SUMMARY OF THE INVENTION

The present invention discloses a compound that causes cancer cell apoptosis this provides an especially advantageous therapy for cancer cell apoptosis and which reduces cell proliferation.

It has already been disclosed that, in addition to dexanabinol being a non competitive NMDA receptor blocker, it has been shown to inhibit NFκB. However, we have now surprisingly established that dexanabinol is capable of actively binding at, or having an indirect effect on, a number of protein sites which were hitherto not known to interact with dexanabinol.

Such protein sites include N-methyl-D-aspartate (NMDA) receptor, Cyclooxygenase-2 (COX-2), Tumour Necrosis factor alpha (TNF-a) and Nuclear factor-kappa B (NFκB). It has previously been reported that Dexanabinol is active at these sites. However, it has not previously been reported that, in addition to its activity at these sites, dexanabinol is also active at the following sites, namely, Cyclin-dependent kinases, e.g. CDK2/A and CDK5/p25, Histone acetyltransferase (HAT) and Farnesyltransferase.

The mechanism of action has now been further investigated and it is now understood that dexanabinol and derivatives thereof have an apoptotic effect on numerous cancer cells. Thus, the apoptosis of cancer cells other than melanoma with dexanabinol, and derivatives thereof, is novel per se.

With the finding that dexanabinol causes cancer cell apoptosis this provides an especially advantageous therapy which reduces cell proliferation and causes cell apoptosis.

In more detail, the known direct and indirect targets of dexanabinol are:

N-Methyl-D-Aspartate (NMDA) Receptor

Dexanabinol was originally developed as a neuroprotective agent. Its neuroprotective action was attributed to its ability to block the NMDA receptor. It blocks NMDA-receptors stereospecifically by interacting with a site close to, but distinct from, that of uncompetitive NMDA-receptor antagonists and from the recognition sites of glutamate, glycine, and polyamines. Unlike some other uncompetitive NMDA receptor antagonists, dexanabinol does not produce psychotropic effects and is generally well tolerated in humans.

Cyclooxygenase-2 (COX-2)

Dexanabinol has anti-inflammatory and antioxidative properties unrelated to its capacity to block NMDA receptors. The anti-inflammatory activity was associated with the ability of dexanabinol to reduce the secretion of PGE2 produced by the enzyme cyclooxygenase-2 (COX-2). COX-2 is one of the cyclooxygenase isoforms involved in the metabolism of arachidonic acid (AA) toward prostaglandins (PG) and other eicosanoids, a family of compounds known to exhibit inflammatory properties and known to be involved in inflammation. Most conventional NSAIDs (non-steroidal anti-inflammatory drugs) inhibit COX activity by modifying the enzyme active site thereby preventing the transformation of the AA substrate to PGE2 (Hinz B. et al., J. Pharm. Exp. Ther. 300: 367-375, 2002). It has been disclosed (WO/2003/077832) that the PGE2 inhibitory activity displayed by dexanabinol does not occur at the level of the COX-2 enzymatic activity, but rather at the level of gene regulation.

Tumour Necrosis Factor Alpha (TNF-a)

Dexanabinol was found to be able to block the production or action of TNF-a. This inhibition most likely occurs at a post-transcriptional level.

Dexanabinol was found to block the production or action of TNF-a, as disclosed in International Patent applications WO 97/11668 and WO 01/98289. It was postulated that the inhibition of the cytokine occurs at a post-transcriptional stage, since in a model of head injury dexanabinol did not affect the levels of TNF-a mRNA (Shohami E. et al., J. Neuroimmuno. 72: 169-77, 1997)

Human TNF-a is first translated into a 27 kd transmembrane precursor protein, which is cleaved into the secreted 17 kd form by TNF-a converting enzyme (TACE). Based on RT-PCR experiments, Shoshany et al. reported that dexanabinol has no significant effect on TNF-a mRNA whereas it significantly reduced the levels of TACE mRNA, supporting the assumption that the drug acts at the level of secretion inhibition.

Nuclear Factor-Kappa B (NFκB)

There is experimental evidence that Dexanabinol inhibits nuclear factor-kappa B (NFκB) indirectly by inhibiting phosphorylation and degradation of IKB2.

Juttler, E et al. (2004) (Neuropharmacology 47(4):580-92.) provided evidence that dexanabinol inhibits NFκB. Dexanabinol inhibits (1) phosphorylation and degradation of the inhibitor of NF-kappaB IkappaBalpha and translocation of NF-kappaB to the nucleus; dexanabinol reduces (2) the transcriptional activity of NF-kappaB and (3) mRNA accumulation of the NF-kappaB target genes tumour necrosis factor-alpha and interleukin-6 (TNF-alpha and IL-6).

The previously unknown targets of dexanabinol are:

Cyclin-Dependent Kinases: CDK2/A and CDK5/p25

Dexanabinol had no significant direct activity against CDK2 and CDK5, when directly assayed. However, we believe that CDKs are affected indirectly, in circumstances where more of the intracellular network that might mediate such effects remains present.

Histone Acetyltransferase (HAT)

Histone acetyl transferase is a known cancer target. No assay data on whether Dexanabinol has activity against this target, however there is predicted activity at this target, which would thus be beneficial

Farnesyltransferase

Farnesyltransferase is a known cancer target. No assay data on whether Dexanabinol has activity against this target, however there is predicted activity at this target.

It is described herein that dexanabinol has effects at more than one protein that are considered to be important in cancers and in cancer therapy. Some of these effects are direct whereas others are indirect. It is of great importance that dexanabinol has effects at numerous targets and this is makes the compound beneficial in a range of cancers.

Cell line data shows that dexanabinol is effective in breast cancer, colon cancer, prostate cancer, non-small cell lung cancer and glioblastoma.

Thus, according to a first aspect of the invention we provide a therapeutic agent capable of having an effect on the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, simultaneously, sequentially or separately. This aspect of the invention is especially advantageous in that, inter alia, it provides a single therapeutic agent for binding the aforementioned proteins.

It will be understood that a particular aspect of the invention provides dexanabinol or a derivative thereof, for having an effect on the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, simultaneously, sequentially or separately.

Thus, according to a further aspect of the invention we provide a therapeutic agent which is capable of having an effect on the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, simultaneously, sequentially or separately for the apoptosis of cancer cells wherein the cancer cells are selected from one or more of primary cancer, breast cancer, colon cancer, prostate cancer, non-small cell lung cancer, glioblastoma, lymphoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

As hereinbefore described, the fact that dexanabinol has direct or indirect effects at the aforementioned protein sites makes it a suitable therapeutic agent for the apoptosis of various cancer cells.

According to a further aspect of the invention we provide dexanabinol, or a derivative thereof, for the apoptosis of cancer in a patient, wherein the cancer is selected from one or more of pancreatic carcinoma, glioblastoma, gastric carcinoma, oesophageal carcinoma, ovarian carcinoma, renal carcinoma and thyroid carcinoma.

According to a further aspect of the invention we provide dexanabinol, or a derivative thereof, for the apoptosis of cancer in a patient, wherein the cancer is selected from one or more of primary cancer, breast cancer, colon cancer, prostate cancer, non-small cell lung cancer, glioblastoma, lymphoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

Thus, the dexanabinol, or a derivative thereof will be a therapeutically effective amount. According to the present invention, a therapeutically effective amount shall mean an apoptotically effective amount.

In addition to the apoptotic effect the dexanabinol, or a derivative thereof, may also provide other cancer treating properties, depending upon, inter alia, the nature of the cancer, such as, inhibition of tumourigenesis, inhibition of cell proliferation, induction of cytotoxicity

It will be understood from the description of the mechanism of action of dexanabinol, and derivatives thereof, that a variety of cancers may be apoptotically treated according the invention. Specific cancers which may be mentioned include, but shall not be limited to, breast cancer, colon cancer, prostate cancer, non-small cell lung cancer, glioblastoma, lymphoma mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer and metastatic cancers. More specific cancers which may be mentioned include cancer selected from one or more of pancreatic carcinoma, glioblastoma, gastric carcinoma, oesophageal carcinoma, ovarian carcinoma, renal carcinoma and thyroid carcinoma. Further specific cancers which may be mentioned include cancer selected from one or more of primary cancer, breast cancer, colon cancer, prostate cancer, non-small cell lung cancer, glioblastoma, lymphoma, and metastatic cancers. Thus, the cancer cells which undergo apoptosis according to the invention may be premalignant, malignant, metastatic, or multidrug-resistant, and combinations thereof. We especially find that dexanabinol, or a derivative thereof, is effective in the apoptosis of metastatic cancer cells.

According to a further aspect of the invention we provide the use of dexanabinol, or a derivative thereof, in the manufacture of a medicament for the apoptosis of cancer in a patient, wherein the cancer is selected from one or more of primary cancer, breast cancer, colon cancer, prostate cancer, non-small cell lung cancer, glioblastoma, lymphoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

In one preferred embodiment of the invention there is provided the use of dexanabinol, or a derivative thereof, in the manufacture of a medicament for the apoptosis of cancer in a patient, wherein the cancer is selected from one or more of pancreatic carcinoma, glioblastoma, gastric carcinoma, oesophageal carcinoma, ovarian carcinoma, renal carcinoma and thyroid carcinoma. In another preferred embodiment of the invention there is provided the use of dexanabinol, or a derivative thereof, in the manufacture of a medicament for the apoptosis of cancer in a patient, wherein the cancer is selected from one or more of primary cancer, breast cancer, colon cancer, prostate cancer, non-small cell lung cancer, glioblastoma, lymphoma, and metastatic cancers.

According to this aspect of the invention we provide the use as hereinbefore described wherein the amount of dexanabinol, or a derivative thereof, administered to a patient is sufficient to achieve a plasma concentration of dexanabinol from 10 to 20 μM.

According to a further aspect of the invention we provide the use as hereinbefore described wherein the amount of dexanabinol, or a derivative thereof, sufficient to achieve a plasma concentration of at least 10 μM of therapeutic agent and is maintained for at least 2 hours in the patient.

According to a yet further aspect of the invention we provide a method of treating cancer wherein the method comprises the apoptosis of the cancer, which comprises administering an apoptotically effective amount of dexanabinol, or a derivative thereof, to a patient in need thereof, wherein the cancer is selected from one or more of primary cancer, breast cancer, colon cancer, prostate cancer, non-small cell lung cancer, glioblastoma, lymphoma, mesothelioma, liver cancer, intrahepatic bile duct cancer, oesophageal cancer, pancreatic cancer, stomach cancer, laryngeal cancer, brain cancer, ovarian cancer, testicular cancer, cervical cancer, oral cancer, pharyngeal cancer, renal cancer, thyroid cancer, uterine cancer, urinary bladder cancer, hepatocellular carcinoma, thyroid carcinoma, osteosarcoma, small cell lung cancer, leukaemia, myeloma, gastric carcinoma and metastatic cancers.

In one preferred embodiment of the invention there is provided a method of treating cancer as hereinbefore described wherein the cancer is selected from one or more of pancreatic carcinoma, glioblastoma, gastric carcinoma, oesophageal carcinoma, ovarian carcinoma, renal carcinoma and thyroid carcinoma. In another preferred embodiment of the invention there is provided a method of treating cancer as hereinbefore described primary cancer, breast cancer, colon cancer, prostate cancer, non-small cell lung cancer, glioblastoma, lymphoma, and metastatic cancers.

The invention especially provides a method of treating cancer wherein the method comprises the apoptosis of the cancer, which comprises the administration of a therapeutically effective amount of an agent capable having either a direct or indirect effect on the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, simultaneously, sequentially or separately. This aspect of the invention is especially advantageous in that, inter alia, it provides a method which comprises the administration of a single therapeutic agent for affecting the aforementioned proteins.

More specifically, the method according to this aspect of the invention comprises administration of a therapeutically effective amount of dexanabinol, or a derivative thereof, to a patient in need of such a therapy.

The method of the invention may comprise the administration of a therapeutically effective amount of dexanabinol, or a derivative thereof, sufficient to inhibit tumourigenesis of a cancer cell.

Alternatively or in addition the method may comprise the administration of a therapeutically effective amount dexanabinol, or a derivative thereof, sufficient to induce cytotoxicity in the cancer cell.

The amount of therapeutic agent, e.g. dexanabinol, which may be administered to a patient, may vary depending upon, inter alia, the nature of the cancer, the severity of the cancer, etc. Thus, for example, the therapeutically effective amount of dexanabinol administered to the patient may be sufficient to achieve a plasma concentration of dexanabinol from 10 to 20 μM.

More specifically, the method may comprise the administration of an effective amount of a therapeutic agent, e.g. dexanabinol, or a derivative thereof, sufficient to achieve a plasma concentration of at least 10 μM of therapeutic agent and is maintained for at least 2 hours in the patient.

We further provide a method of simultaneously, sequentially or separately effecting the proteins N-methyl-D-aspartate (NMDA), Cyclooxygenase-2 (COX-2), Tumour Necrosis factor alpha (TNF-a), Nuclear factor-kappa B (NFκB), Cyclin-dependent kinases, e.g. CDK2/A and CDK5/p25, Histone acetyltransferase (HAT) and Farnesyltransferase, which comprises the administration of an effective amount of dexanabinol, or a derivative thereof.

According to a yet further aspect of the invention we provide a pharmaceutical composition comprising dexanabinol, or a derivative thereof, wherein the amount of dexanabinol, or a derivative thereof, present is sufficient to achieve a plasma concentration of dexanabinol from 10 to 20 μM.

We further provide a pharmaceutical composition comprising dexanabinol, or a derivative thereof, wherein the amount of dexanabinol, or a derivative thereof, sufficient to achieve a plasma concentration of at least 10 μM of dexanabinol and is maintained for at least 2 hours in the patient.

The present invention contemplates that the cancer cells may be premalignant, malignant, primary, metastatic or multidrug-resistant

Alternatively, the treatment of the cancer may comprise the inhibition of tumourigenesis of a cancer cell by contacting the cell with an effective amount of dexanabinol, or a derivative thereof. Inhibition of tumourigenesis may also include inducing cytotoxicity and/or apoptosis in the cancer cell.

Furthermore the method of the invention is advantageous because, inter alia, it shows reduced toxicity, reduced side effects and/or reduced resistance when compared to currently employed chemotherapeutic agents.

It is further contemplated that a second therapeutic agent may be provided in combination with dexanabinol, or a derivative thereof, to a cancer cell for treatment and/or prevention of the cancer. The second therapeutic agent may comprise a chemotherapeutic agent, immunotherapeutic agent, gene therapy or radio therapeutic agent. When a second therapeutic agent is included in the treatment according to the invention, the second therapeutic agent may be administered with the dexanabinol, or a derivative thereof, separately, simultaneously or sequentially.

Although a variety of second or additional therapeutic agents may be used in conjunction with dexanabinol, or a derivative thereof. However, preferably, the second or additional therapeutic agent may be selected from the group consisting of: a chemotherapeutic agent, an immunotherapeutic agent, a gene therapy agent, and a radiotherapeutic agent.

The term “derivative” used herein shall include any conventionally known derivatives of dexanabinol, such as, inter alia, solvates. It may be convenient or desirable to prepare, purify, and/or handle a corresponding solvate of the compound described herein, which may be used in any one of the uses/methods described. The term solvate is used herein to refer to a complex of solute, such as a compound or salt of the compound, and a solvent. If the solvent is water, the solvate may be termed a hydrate, for example a mono-hydrate, di-hydrate, tri-hydrate etc, depending on the number of water molecules present per molecule of substrate. The term derivative shall especially include a salt. Suitable salts of dexanabinol are well known and are described in the prior art. Salts of organic and inorganic acids and bases that may be used to make pharmaceutically acceptable salts. Such acids include, without limitation, hydrofluoric, hydrochloric, hydrobromic, hydroiodic, sulphuric, nitric, phosphoric, citric, succinic, maleic, and palmitic acids. The bases include such compounds as sodium and ammonium hydroxides. Those skilled in the art are familiar with quaternizing agents that can be used to make pharmaceutically acceptable quaternary ammonium derivatives of dexanabinol. These include without limitation methyl and ethyl iodides and sulphates.

Dexanabinol and derivatives and/or combinations thereof are known per se and may be prepared using methods known to the person skilled in the art or may be obtained commercially. In particular, dexanabinol and methods for its preparation are disclosed in U.S. Pat. No. 4,876,276.

According a further aspect of the invention we provide the use of dexanabinol, or a derivative thereof, or a method as hereinbefore described wherein the dexanabinol, or a derivative thereof, is administered in admixture with a pharmaceutically acceptable adjuvant, diluent or carrier.

The dexanabinol, or a derivative thereof, may be administered in a variety of ways depending upon, inter alia, the nature of the cancer to be treated. Thus, the dexanabinol, or a derivative thereof, may be administered topically, transdermally, subcutaneously, intravenously, or orally.

We especially provide a use of dexanabinol, or a derivative thereof, or a method of treatment, which comprises the topical administrable of dexanabinol, or a derivative thereof.

Thus, in the use, method and/or composition of the invention of the compound may be put up as a tablet, capsule, dragee, suppository, suspension, solution, injection, e.g. intravenously, intramuscularly or intraperitoneally, implant, a topical, e.g. transdermal, preparation such as a gel, cream, ointment, aerosol or a polymer system, or an inhalation form, e.g. an aerosol or a powder formulation.

Compositions suitable for oral administration include tablets, capsules, dragees, liquid suspensions, solutions and syrups;

Compositions suitable for topical administration to the skin include creams, e.g. oil-in-water emulsions, water-in-oil emulsions, ointments, gels, lotions, unguents, emollients, colloidal dispersions, suspensions, emulsions, oils, sprays, foams, mousses, and the like. Compositions suitable for topical application may also include, for example, liposomal carriers made up of lipids or special detergents.

Examples of other adjuvants, diluents or carriers are:

for tablets and dragees—fillers, e.g. lactose, starch, microcrystalline cellulose, talc and stearic acid; lubricants/glidants, e.g. magnesium stearate and colloidal silicon dioxide; disintegrants, e.g. sodium starch glycolate and sodium carboxymethylcellulose;

for capsules—pregelatinised starch or lactose;

for oral or injectable solutions or enemas—water, glycols, alcohols, glycerine, vegetable oils;

for suppositories—natural or hardened oils or waxes.

It may be possible to administer the compound or derivatives and/or combination thereof or any combined regime as described above, transdermally via, for example, a transdermal delivery device or a suitable vehicle or, e.g. in an ointment base, which may be incorporated into a patch for controlled delivery. Such devices are advantageous, as they may allow a prolonged period of treatment relative to, for example, an oral or intravenous medicament.

Examples of transdermal delivery devices may include, for example, a patch, dressing, bandage or plaster adapted to release a compound or substance through the skin of a patient. A person of skill in the art would be familiar with the materials and techniques which may be used to transdermally deliver a compound or substance and exemplary transdermal delivery devices are provided by GB2185187, U.S. Pat. No. 3,249,109, U.S. Pat. No. 3,598,122, U.S. Pat. No. 4,144,317, U.S. Pat. No. 4,262,003 and U.S. Pat. No. 4,307,717.

The invention will now be illustrated by way of example only.

### DETAILED DESCRIPTION

### Example 1

In Vitro Assay to Evaluate the Effect on Apoptosis of Dexanabinol in Cell Lines

Methods

Assay was performed at a 24 hour timepoint on 3 melanoma lines (A375, G-361, WM266-4) 2 breast cancer lines (MCF7, MDA-MB-231), fibroblast (46BR.1G1), colon cancer (HCT116), prostate cancer (PC-3), glioblastoma (U373) and non-small cell lung cancer (NSCLC) (DMS-114)

The above cell lines were maintained in RPMI 1640 culture medium (Sigma, UK) containing 10% (v/v) heat inactivated foetal bovine serum (Sigma, UK) and 2 mM L-glutamate at 37° C. in 5% humidified CO₂. Cells were harvested, washed, re-suspended into growth medium and counted (Beckman-Coulter Vi-CELL XR). Cells were plated onto the middle 240 wells of 384 tissue culture plates at 1.6×lO⁵ to 2.4×lO⁵ cells/ml in 12.5 μl/well aliquots. 50 μl of growth media was aliquoted into the outer wells. 2 plates were prepared per cell line. Plates were incubated overnight at 37° C., in 5% humidified CO₂.

Dexanabinol was prepared in growth medium at 2 times the final assay concentration at 125, 31.3, 7.81, 2.00, 0.49, 0.12, 0.031 and O.OO8 μM (DMSO concentration was kept constant across the dilution range at 0.5%).

Cisplatin was used as a positive control. The final assays concentrations were 10, 2.5, 0.63, 0.156, 0.039, 0.010, 0.002 and 0.0006 μg/ml. 12.5 μl per well of dexanabinol or cisplatin dilutions were added to the plates in replicates of 6. 12.5 μl of growth media was added to the media control wells. The plates were incubated for 24 hours at 37° C., in 5% humidified CO₂.

Caspase 3/7 levels were assessed by Apo-ONE® Homogeneous Caspase-3/7 assay kit. Fluorescence was measured using a FlexStation® II³⁸⁴ plate reader at 1, 2, 3 and 4 hours after addition of the caspase substrate. The 4 hour readings were used for analysis.

The cell viability assay was performed in parallel on the same plate for each line using CellTiter-Blue® (Promega) reagent. Briefly, 25 μl of CellTiter-Blue® (Promega) reagent was added to each well. The plates were shaken for 1 minute at 500 rpm and then incubated at 37° C., 5% CO₂ for 4 hours. Fluorescence was measured using a FlexStation® II³⁸⁴ plate reader (570 nm excitation wavelength, 600 nm emission wavelength, 590 nm cut-off.) The plots showing the cytotoxic effect of dexanabinol and cisplatin are shown as an overlay on the same graph.

Results

The induction of apoptosis in A375, G-361, WM266-4, MCF7, MDA-MB-231, 46BR.1G1, HCT116, PC-3, U373 and DMS-114 cells following 24 hours incubation with either cisplatin or dexanabinol is shown in FIGS. 1-10 respectively and summarized in Table 1. In addition the assessment of cell viability as measured by the CellTiter-Blue® assay indicating cytotoxicity is also shown.

Cisplatin was used as a positive control and a cytotoxic response was observed in all cell lines with an approximate IC₅₀ value of 5-20 μg/ml, except for U373MG and MDA-MB231 which showed some degree of resistance to its cytotoxic effect. Inadequate dose responses were observed for DMS114 and PC3 cells, therefore the IC₅₀ values could not be determined. The induction of apoptosis was not as easily quantified due to either inadequate dose curves (G-361, WM266-4 & PC3) or poor caspase 3/7 induction (MDA-MB231, MCF-7, HCT116, DMS114 & U373MG). Overall, the three melanoma cell lines (A375, G-361 and WM266-4), colon cancer line (HCT116) and the fibroblast line, 46BrlG1, were the most sensitive to the cytotoxic effects of cisplatin, inducing both an increase in apoptosis and a decrease in cell viability.

Dexanabinol induced a cytotoxic response with IC₅₀ values in the range of 10-25 μM in the majority of cell lines. The induction of apoptosis was not quantified for all cell lines due to either inadequate dose response curves (A375, G-361, PC3, 46Br.1G1 & DMS-114) or non-responding cells (MCF-HCT116 & U373MG). A peak response in apoptosis occurred at 2.5 μM and dropped at the highest concentration of 10 μM possibly due to cell lysis and loss. Overall, the three melanoma cell lines (A375, G-361 and WM266-4), 2 breast cancer lines (MDA-MB231 and MCF7) and the prostate line (PC3M) were the most sensitive to dexanabinol with DMS114 and U373 being the least sensitive.

TABLE 1
Results: Summary of data

| | Cisplatin | Dexanabinol |
|---|---|---|
| Cell line | ↓ Viability IC₅₀ (μM) | ↓ Viability IC₅₀ (μM) |
| Melanoma | | |
| A375 | 21.8** | 19.16** |
| G-361 | 18.00** | 10.97*** |
| WM266-4 | 62.00* | 20.87** |
| Breast cancer | | |
| MCF7 | 40.60** | 16.19*** |
| MDA-MB-231 | NR* | Approx 10-50** |
| Colon cancer | | |
| HCT116 | 29.50** | 22.34*** |
| Prostate cancer | | |
| PC-3 | Approx 58.00* | 19.91*** |
| NSCLC | | |
| DMS-114 | Approx 40.20* | Approx 10-50* |
| Glioblastoma | | |
| U373 | NR* | Approx 10-50* |
| Fibroblast | | |
| 46Br.1G1 | 21.50** | 23.09*** |

ND—EC/IC₅₀ not determined due to inadequate dose response curve
NR—No response
observed
Rank
*weak apoptosis induction and decrease in proliferation (<35%)
**moderate apoptosis induction and decrease in proliferation (35-70%)
***good apoptosis induction and decrease in proliferation (>70%)

### SUMMARY

In previous studies, as detailed in WO '700, dexanabinol decreased growth in melanoma cell lines (A375, Malme-3M, UACC62) with an IC₅₀ value of in the range of 10-20 μM. The objective of this study was to determine if dexanabinol induced apoptosis in a panel of cancer cell lines and a human fibroblast line in order to elucidate a potential mechanism of action. In addition to apoptosis, cell viability was also assessed in parallel.

Cisplatin, a standard of care agent used in the clinic to treat a range of cancers, including gastrointestinal cancers and glioblastomas, was used as a positive control and induced cytotoxic effects in the majority of cell lines, except for U373MG, DMS114, PC3 and MDA-MB231, which showed some degree of resistance. In those cell lines responding to cisplatin, the decrease in viability corresponded to an increase in apoptosis, except for MCF7, which is reported to be Caspase 3 deficient, thus apoptosis may be underestimated in this cell line.

The test agent, dexanabinol, showed a pro-apoptotic effect which completely coincided with its effect on cell number in a similar manner to that seen with the DNA-chelating agent, cisplatin. The effects were shown at concentrations of 10 μM upwards.

Dexanabinol produced a dose-dependent decrease in cell viability in all cell lines at a concentration>10⁻⁵M, but apoptosis did not always correspond to this pattern with a peak response occurring at a concentration of 2.5 μM and then disappearing at 10 μM.

However, this may have been due to the 100% loss in cell viability at the highest concentration which resulted in insufficient cells to assay the apoptotic event. The most sensitive cell lines appeared to be:
Human melanomas: WM366-4, G-361
Human breast: MDA-MB-231
Human prostate: PC3

### Example 2

MTT Assay
Evaluation of dexanabinol plus a positive control
Screening against multiple cell lines selected from different tumour types, e.g.:

| Cancer | Cell line |
|---|---|
| acute myeloid leukaemia | MV4-11 |
| renal cell carcinoma | 786-0 |
| multiple myeloma | OPM-2 |
| pancreatic cancer | PANC-1 |
| pancreatic cancer | BxPC-3 |
| acute lymphoblastic leukaemia | MOLT-4 |
| ovarian cancer | A2780 |
| chronic myeloid leukaemia | K-562 |
| gastric cancer | MKN-45 |
| gastric cancer | NCI-N87 |
| acute promyelocytic leukaemia | HL-60 |
| small cell lung cancer | NCI-H69 |
| small cell lung cancer | NCI-H526 |
| medullary thyroid carcinoma | TT |
| oesophageal carcinoma | OE33 |
| osteosarcoma | SJSA-1 |
| anaplastic thyroid cancer | 8505C |
| glioblastoma | U87MG |
| glioblastoma | SF-295 |
| diffuse large B cell lymphoma | WSU-DLCL2 |
| hepatocellular carcinoma | Hep3B |
| hepatocellular carcinoma | Hep G2 |

Specific Aim 1: IC₅₀ Value Determination of Single Agents.

The human tumour cells will be placed in a 96-well microculture plate (Costar white, flat bottom #3917) in a total volume of 90 μl/well. After 24 hours of incubation in a humidified incubator at 37° C. with 5% CO₂ and 95% air, 10 gil of 10×, serially diluted test agents in growth medium will be added to each well. After 96 total hours of culture in a CO₂ incubator, the plated cells and Cell Titer-Glo (Promega #G7571) reagents will be brought to room temperature to equilibrate for 30 minutes. 100 μl of Cell Titer-Glo® reagent will be added to each well. The plate will be shaken for 2 minutes and then left to equilibrate for 10 minutes before reading luminescence on the Tecan GENios microplate reader.

Percentage inhibition of cell growth will be calculated relative to untreated control wells. All tests will be performed in duplicate at each concentration level.

The IC₅₀ value for the test agents will be estimated using Prism 3.03 by curve-fitting the data using the following four parameter-logistic equation:

$$Y = \frac{Top - Bottom}{1 + {( X / {IC}_{50} )}^{n}} + Bottom$$

where Top is the maximal % of control absorbance, Bottom is the minimal % of control absorbance at the highest agent concentration, Y is the % of control absorbance, X is the agent concentration, IC₅₀ is the concentration of agent that inhibits cell growth by 50% compared to the control cells, and n is the slope of the curve.

### Example 3

Xenograft Study
Cells: Dependent on outcome of in vitro studies
Mice: athymic female mice, 6-8 weeks old
Tumours: single flanks implanted with 5 million cells with matrigel.
Drugs: dexanabinol, ip, once weekly×4 weeks Cisplatin or taxol, ip once weekly×4 weeks

GROWTH CURVE: choose the mice with the most similar tumour size, around 150 mm³

Treatment groups: (6 mice/group):
1. vehicle alone i.p. once weekly 4 weeks
2. dexanabinol, i.p, once weekly×4 weeks
3. Cisplatin, ip once weekly×4 weeks
4. dexanabinol, i.p, once weekly+Cisplatin, ip once weekly×4 weeks

Tumour measurements: Two times a week until mice are sacrificed and tumours collected

Weight measurements: at least twice weekly.

1. A method of treating cancer comprising administration of a therapeutically effective amount of dexanabinol, or a derivative thereof, to a patient in need of such a therapy, wherein the cancer is selected from one or more of pancreatic carcinoma, glioblastoma, gastric carcinoma, ovarian carcinoma & renal carcinoma.

2. A method according to claim 1 wherein the cancer is pancreatic carcinoma.

3. A method according to claim 1 wherein the cancer is glioblastoma.

4. A method according to claim 1 wherein the cancer is gastric carcinoma.

5. A method according to claim 1 wherein the cancer is ovarian carcinoma.

6. A method according to claim 1 wherein the cancer is renal carcinoma.

7. A method according to claim 1 wherein the method comprises administration of a therapeutically effective amount dexanabinol, or a derivative thereof, sufficient to induce apoptosis of the cancer.

8. A method according to claim 1 wherein the method comprises administration of a therapeutically effective amount of dexanabinol, or a derivative thereof, sufficient to inhibit tumourigenesis of a cancer cell.

9. A method according to claim 1 wherein the method comprises administration of a therapeutically effective amount dexanabinol, or a derivative thereof, sufficient to induce cytotoxicity in the cancer cell.

10. A method according to claim 1 wherein the method comprises administration of dexanabinol, or a derivative thereof, wherein the amount administered to a patient is sufficient to achieve a plasma concentration of dexanabinol from 10 to 20 μM.

11. A method according to claim 1 wherein the method comprises administration of an effective amount of dexanabinol, or a derivative thereof, sufficient to achieve a plasma concentration of at least 10 μM of therapeutic agent and is maintained for at least 2 hours in the patient.

12. A method according to claim 1 wherein the cancer cells are premalignant, malignant, metastatic or multidrug-resistant and combinations thereof.

13. A method according to claim 1 which comprises administration of dexanabinol, or a derivative thereof, in combination with another cancer treating therapeutic agent a derivative thereof, separately, simultaneously or sequentially.

14. A method according to claim 1 in combination with another cancer treating therapeutic agent wherein the other cancer treating therapeutic agent is suitable for inhibition of tumourigenesis, inhibition of cell proliferation, or induction of cytotoxicity.

15. A method according to claim 1 wherein the other therapeutic agent comprises a chemotherapeutic agent, immunotherapeutic agent, gene therapy or radio therapeutic agent.

16. A method according to claim 1 wherein the dexanabinol, or a derivative thereof, is administered topically, transdermally, subcutaneously, intravenously, or orally.

17. A pharmaceutical composition comprising dexanabinol, or a derivative thereof, wherein the amount of dexanabinol, or a derivative thereof, present is sufficient to achieve a plasma concentration of dexanabinol from 10 to 20 μM.

18. A pharmaceutical composition comprising dexanabinol, or a derivative thereof, wherein the amount of dexanabinol, or a derivative thereof, sufficient to achieve a plasma concentration of at least 10 μM of dexanabinol and is maintained for at least 2 hours in the patient.
